(19) United States
(12) Patent Application Publication
(10) Pub. No.: US 2025/0312581 A1
(43) Pub. Date: Oct. 9, 2025

(54) DILATOR POSITIONING ASSEMBLY

(71) Applicant: THREE PEAKS MEDICAL PTY LTD, Norwood (AU)

(72) Inventors: Simon BELCHER, Panorama (AU); Adam DI FIORE, West Hindmarsh (AU)

(73) Assignee: THREE PEAKS MEDICAL PTY LTD, Norwood (AU)

(21) Appl. No.: 18/862,977

(22) PCT Filed: May 11, 2023
(86) PCT No.: PCT/IB2023/054875

§ 371 Date: Nov. 5, 2024

(30) Foreign Application Priority Data
May 16, 2022 (WO) PCT/AU2022/050466

(51) Int. Cl. A61M 29/02; A61B 17/34

(52) U.S. Cl. CPC A61M 29/02; A61B 17/3468; A61M 2029/025; A61M 2205/0216

(57) ABSTRACT

Vascular dilator positioning assemblies and vascular dilator positioning systems are described herein. Also provided herein are methods for the use and assembly of these vascular dilator positioning assemblies and systems. Further, these assemblies and systems include a grip portion that has a wall and an actuator for positioning the dilator tip relative to a sheath.

### TECHNICAL FIELD

The technical field generally relates to vascular dilators, introducer sheaths and components thereof for insertion into the body to provide intravascular access to various medical devices. This includes but is not limited to all arterial and vasculature access, abdominal and thoracic cavities, cerebrospinal, genito-urinary and gynaecological, upper gastrointestinal and colorectal procedures.

Embodiments generally provide systems and methods for positioning a dilator head relative to an introducer sheath to reduce the gap therebetween.

### BACKGROUND

Vascular access devices, including vascular introducer sheaths and vascular dilators, are now very commonplace in many intravascular procedures, such as transcatheter aortic valve replacement (TAVR), angioplasty and stenting. They generally facilitate access to the vascular system for the introduction of removable devices such as wires, balloons, pressure transducers and for the introduction and placement of implantable devices such as mechanical aortic valves and stents.

Vascular access devices are now typically inserted into the patient's vessel through the skin percutaneously. Practice away from cutting the skin with a scalpel to expose the vessel for insertion has led to the safer and more frequent use of vascular access devices. Percutaneous insertion techniques involve the insertion of a needle or similar puncture device into the skin without first cutting the skin, to expose the vessel. Vascular access devices are advanced through the skin and into the vessel through the puncture. For patients, percutaneous techniques reduce blood loss and reduce the likelihood of human error that may occur while making scalpel incisions.

During percutaneous procedures, an introducer sheath is typically positioned at an entry point to a patient vessel and pushed through the vessel until the sheath is firmly seated where it is required within the patient. The proximal end of the introducer sheath protrudes outside of the patient's body to provide an entry point for the subsequent insertion of additional or other intravascular devices. Frequently, a dilator is placed within the introducer sheath and is pushed through the vessel opening with the introducer sheath to gradually open the vessel puncture site and allow the sheath to enter while minimising vessel trauma.

The percutaneous entry point is typically much smaller than the diameter of the introducer sheath and some force is required to stretch the patient's skin at the percutaneous opening to pass the vascular access device into the vessel. Therefore, any device having a groove, notch, or angular edge is likely to create a catch point where the skin may catch and tear, thereby causing site trauma and delay in recovery for the patient.

Typical introducer sheaths therefore have a tapered distal end to provide a smooth transition to the dilator end and tip when used to open up the vasculature, to allow the sheath to enter without excessive trauma. Ideally, to avoid trauma, the transition between the dilator tip and the sheath distal end is 0.1 mm or less. But this is not always achieved. Where the dilator is significantly longer than the sheath, the sheath and dilator assembly tend to be more tolerant of small manufacturing variations, particularly in variation of length of either the dilator or sheath, but for shorter lengths this is not always achieved.

Also, not all sheath and dilator types are capable of tolerating manufacturing variations. For introducer sheaths having multiple layers, the insertion of a tapered tip into an introducer sheath with layers that slide against each other is difficult if not impossible. To prevent the excessive movement of layers a stepped gap is frequently present between a typical dilator tip and the sheath. To overcome this, a dilator tip with a rounded arrow-head distal end shape is used to provide a smooth transition between dilator and sheath.

Where arrow-head type dilator tips are used, there remains a potential gap between the arrow-head dilator and sheath arising from small manufacturing variations, which create a catch point for skin and flesh during insertion of the dilator and sheath into the body.

Therefore, a mechanism is needed to position a dilator tip relative to an introducer sheath to minimise a catch point created by gaps or edges at the introducer sheath and dilator transition area.

### SUMMARY OF INVENTION

Embodiments of the invention relate to a vascular dilator positioning assembly comprising; a grip portion comprising a wall having an outer wall surface and an inner wall surface, the wall having a proximal edge defining a proximal opening, a distal edge defining a distal opening and a lumen formed therethrough, a user-operatable actuator located about the proximal edge, and a vascular dilator comprising an elongated tube attached to the actuator at a proximal end and a dilator tip at a distal end, wherein the actuator is configured to move the dilator tip distally from a retracted position to an extended position while the actuator is activated and is configured to return the dilator tip to the retracted position when the actuator stops being activated.

As used herein the terms ‘proximal’ and ‘distal’ are to be understood as opposite terms being relative to the direction of use of the article to which they refer. The term ‘distal’ is to be understood to refer to the end of the article first entering the patient, and the term ‘proximal’ is to be understood to refer to the end opposite the ‘distal’ end. For example, the ‘distal end’ may be nearest to the dilator tip such that in usage the distal end is closest to the patient and the proximal end is closest to the user, the ‘proximal’ end also being nearest to the actuator.

In embodiments, the actuator has a button located about the proximal edge and an actuator core configured to engage with the wall, wherein the actuator is configured to move the dilator tip distally from the retracted position to the extended position upon the application of force to the button and is configured to return the dilator tip to the retracted position upon the release of force to the button.

Preferably, the actuator core comprises a plug portion having a width spanning a width of grip portion lumen. The plug portion may be configured as a plate or bung and may be configured to plug the grip portion lumen. The plug portion may guide the movement of the dilator tip.

Dilator tips are preferably substantially conical with a taper towards a point at the distal end. More preferably, dilator tips are arrowhead shaped and comprise two substantially conical portions wherein the first conical portion tapers outwards from the proximal end towards a maximum width and the second portion tapers inwards towards a rounded tip at the distal end.

Alternatively, the dilator tip may be of substantially uniform width along its length with a flat proximal end surface and a rounded tip at its distal end.

Sheaths for use with embodiments of the invention preferably comprise a semi-rigid layer to prevent significant longitudinal crumpling of the sheath but may alternatively comprise a semi-rigid element configured to substantially maintain the length of the sheath. Configurations including single layer and multi-layer sheaths may be suitable for this invention.

In some embodiments, the actuator core is configured to occupy a space within the grip lumen.

In embodiments, the vascular dilator positioning assembly comprises a resilient biasing element for biasing a dilator tip with respect to the wall. In further embodiments, the resilient biasing element is a spring having a spring rate of between about 0.1 N/mm and 10 N/mm.

Preferably, the installed force at maximum deflection is between about 0.1 N and 10 N. More preferably, the spring rate of the resilient biasing element is between 1 N/mm and 3 N/mm and the installed force at maximum deflection ranges from 0.5 N to 6.5 N. Most preferably, the spring rate of the resilient biasing element is approximately 0.6 N/mm.

In alternative embodiments, the resilient biasing element may be an air pocket which compresses upon applied force and is biased towards expansion, a rubber stopper, an elastic strip, or repulsive magnets having a force per distance of between about 0.1 N/mm and 10 N/mm or an installed force at maximum deflection range of between 0.1 N and 10 N.

In some embodiments, the inner wall surface comprises one or more protrusions. The protrusions are preferably configured such that the actuator core, having a plug portion, and the one or more protrusions form a space therebetween for maintaining the compressible biasing element therein.

In further embodiments, a portion of the actuator core is shaped to occupy a space across a width of the grip lumen thereby enabling the actuator to slide within the grip lumen, and the resilient biasing element occupies a space substantially defined by an outer surface of the actuator core and the inner surface of the wall.

Embodiments of the invention may further comprise; a dilator sheath circumferentially surrounding the elongated tube having a proximal sheath end and a distal sheath end wherein the actuator is configured to move the dilator tip distally from a retracted position to an extended position upon the application of force to the button to widen a gap between the dilator sheath and the dilator tip and is configured to partially return the dilator tip to an intermediary resting position upon the release of force from the button thereby closing the gap between the dilator sheath and the dilator tip.

The application of force on the button is preferably a user controlled thumb or finger press wherein the user receives tactile feedback from the resilient biasing element and is able to control the release of force for a controlled retraction of the dilator tip.

The retracted position is preferably such that the dilator tip is partially within the dilator sheath. The extended position is preferably such that the dilator tip has fully exited the dilator sheath to expose its proximal end and create a gap therebetween. The intermediary resting position is preferably between the retracted position and the extended position and is defined by the point at which a surface of the dilator tip abuts the dilator sheath.

In further embodiments, the dilator sheath comprises a semi-rigid inner layer and an elastomeric outer layer and the dilator tip is configured to abut the semi-rigid inner layer at the intermediary position thereby providing a substantially smooth exterior surface across the elastomeric outer layer and the dilator tip.

The abutment of the dilator tip against the semi-rigid inner layer may be at any surface of the dilator tip. Preferably, the dilator tip abuts the semi-rigid inner layer in a tapered surface of a proximal conical portion but alternatively the semi-rigid inner layer may abut a flat surface at the proximal end of the dilator tip or protuberances about the dilator tip.

In further preferred embodiments the resilient biasing element is a spring having a spring rate of between about 0.1 N/mm and 10 N/mm.

Embodiments of the invention relate to a vascular dilator positioning system comprising; a grip portion comprising a wall having an outer wall surface and an inner wall surface, the wall having a proximal edge defining a proximal opening, a distal edge defining a distal opening and a lumen formed therethrough, a user-operatable actuator located about the proximal edge, and a vascular dilator comprising an elongated tube attached to the actuator at a proximal end and a dilator tip at a distal end, wherein the actuator is configured to move the dilator tip distally from a retracted position to an extended position while the actuator is activated and is configured to return the dilator tip to the retracted position when the actuator stops being activated.

In embodiments, the vascular dilator positioning assembly comprises a resilient biasing element for biasing a dilator tip with respect to the wall. In further embodiments the resilient biasing element is a spring having a spring rate of between about 0.1 N/mm and 10 N/mm.

In embodiments, the actuator has a button located about the proximal edge and an actuator core configured to engage with the wall, wherein the actuator is configured to move the dilator tip distally from the retracted position to the extended position upon the application of force to the button and is configured to return the dilator tip to the retracted position upon the release of force to the button.

Further embodiments may comprise a dilator sheath circumferentially surrounding the elongated tube having a proximal sheath end and a distal sheath end wherein the actuator is configured to move the dilator tip distally from a retracted position to an extended position upon the application of force to the button to widen a gap between the dilator sheath and the dilator tip and is configured to partially return the dilator tip to an intermediary resting position upon the release of force from the button thereby closing the gap between the dilator sheath and the dilator tip.

In some embodiments the dilator sheath comprises a semi-rigid inner layer and an elastomeric outer layer and the dilator tip is configured to abut the semi-rigid inner layer at the intermediary resting position thereby providing a substantially smooth exterior surface across the elastomeric outer layer and the dilator tip.

Embodiments of the invention relate to a method of use of a vascular dilator positioning assembly comprising the steps of: obtaining a grip portion comprising a wall having an outer wall surface and an inner wall surface, the wall having a proximal edge defining a proximal opening, a distal edge defining a distal opening and a lumen formed therethrough and an user-10 operatable actuator located about the proximal edge, attaching a vascular dilator comprising an elongated tube to the actuator at a proximal end and a dilator tip at a distal end, placing a dilator sheath circumferentially around the elongated tube having a proximal sheath end and a distal sheath end, and activating the actuator to move the dilator tip distally from a retracted position to an extended position to widen a gap between the dilator sheath and the dilator tip, and stop activating the actuator to return the dilator tip to the retracted position.

In embodiments, the method comprises the step of assembling the grip portion, the actuator and a resilient biasing element for biasing a dilator tip with respect to the wall.

In embodiments, the actuator has a button located about the proximal edge and an actuator core configured to engage with the wall. The step of activating the actuator comprises applying force to the button to move the dilator tip distally from a retracted position to an extended position to widen a gap between the dilator sheath and the dilator tip. The step of stop activating the actuator comprises releasing force to the button to partially return the dilator tip to an intermediary resting position thereby closing the gap between the dilator sheath and the dilator tip.

In further methods the dilator sheath comprises a semi-rigid inner layer and an elastomeric outer layer and the dilator tip is configured to abut the semi-rigid inner layer at the intermediary resting position thereby providing a substantially smooth exterior surface across the elastomeric outer layer and the dilator tip; and the resilient biasing element is a spring having a spring rate of between about 0.1 N/mm and 10 N/mm.

Embodiments of the invention relate to a method of assembling a vascular dilator positioning system comprising the steps of: obtaining a grip portion comprising a wall having an outer wall surface and an inner wall surface, the wall having a proximal edge defining a proximal opening, a distal edge defining a distal opening and a lumen formed therethrough, obtaining an actuator located about the proximal edge, attaching a vascular dilator comprising an elongated tube to the actuator core at a proximal end and a dilator tip at a distal end, and placing a dilator sheath circumferentially around the elongated tube having a proximal sheath end and a distal sheath end.

In embodiments, the method comprises the step of engaging the grip portion with the actuator and a resilient biasing element for biasing a dilator tip with respect to the wall.

In further methods the resilient biasing element is a spring having a spring rate of between about 0.1 N/mm and 10 N/mm.

Broad embodiments of the invention now will be described with reference to the accompanying drawings together with the Examples and the preferred embodiments disclosed in the detailed description. The invention may be embodied in many different forms and should not be construed as limited to the embodiments described herein. These embodiments are provided by way of illustration only such that this disclosure will be thorough, complete and will convey the full scope and breadth of the invention.

### BRIEF DESCRIPTION OF THE FIGURES

FIG. 1 provides a front view of a prior art dilator assembly showing a prior art introducer sheath and collar in dotted line transparency. A detailed view of portion A provided in Detail A.

FIG. 2 provides front section views of the prior art dilator and sheath assembly shown in Detail A of FIG. 1. FIG. 2a shows the prior art dilator tip during initial insertion into an incision. FIG. 2b shows the prior art dilator tip after insertion. FIG. 2c shows the prior art sheath during insertion.

FIG. 3 provides a side perspective view of a dilator according to embodiments of the invention. Detail B provides an enlarged view of the dilator tip.

FIG. 4 provides a side perspective exploded view of a dilator and introducer sheath assembly according to embodiments of the invention.

FIG. 5 provides a front section view of a dilator and introducer sheath assembly according to embodiments of the invention. A detailed view of portion C is provided in Detail C.

FIG. 6 provides detailed side perspective views of the dilator and introducer sheath proximal ends according to embodiments of the invention. FIG. 6a details the dilator and FIG. 6b details the dilator and introducer sheath assembly.

FIG. 7 provides front section views of a dilator and introducer sheath assembly according to embodiments. FIG. 7a provides detail of the dilator tip prior to actuation of the biasing element. A detailed view of portion D is provided in Detail D. FIG. 7b provides detail of the dilator tip at full actuation of the biasing element. A detailed view of portion E is provided in Detail E. FIG. 7c provides detail of the dilator tip after deactuation of the biasing element. A detailed view of portion F is provided in Detail F.

FIG. 8 provides front section views of a dilator and sheath assembly according to embodiments of the invention shown in Detail F of FIG. 7c. FIG. 8a shows the dilator tip during initial insertion into an incision. FIG. 8b shows the dilator tip after insertion. FIG. 8c shows the sheath during insertion.

FIG. 9 provides a flow chart of a method of use for a dilator positioning assembly according to embodiments of the invention.

Several embodiments of the invention are described in the following examples.

### DETAILED DESCRIPTION

### Prior Art

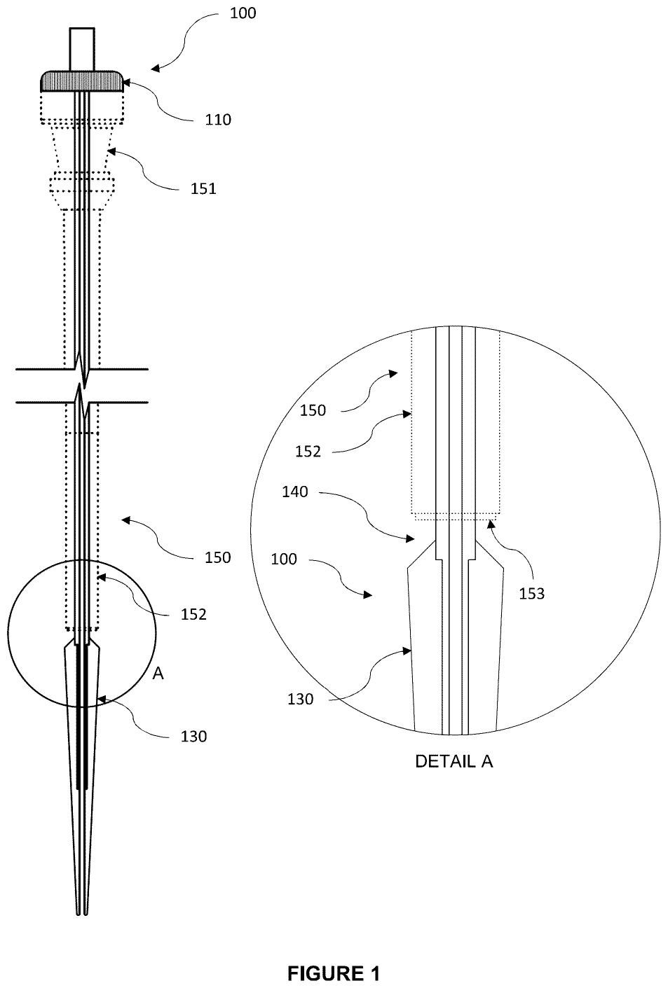

FIG. 1 provides a front view of a prior art dilator 100 assembly showing a prior art introducer sheath 150 in dotted line transparency. Detail A provides an enlarged front view of the dilator tip 130 and sheath. Dilator 100 is a tubular device for insertion into a vessel opening to reduce trauma while dilating the vessel for introduction of the introducer sheath 150.

In many prior art dilator and introducer sheath assemblies of this kind, the design lengths of both dilator 100 and introducer sheath 150 are such that when dilator hub 110 is attached to collar 151, the distal end 153 of sleeve 152 is nominally located in contact with the proximal end of tapered head 130 to provide a substantially smooth transition therebetween.

Detail A illustrates a detail view of the distal end 153 of sleeve 152 and the proximal end of tapered head 130 of introducer sheath 150 and dilator 100 respectively. While the design lengths of both dilator 100 and introducer sheath 150 nominally locate tapered head 130 at the distal end 153 of sleeve 152, manufacturing error in both the dilator 100 and introducer sheath 150 can lead to a gap 140 therebetween. The presence of gap 140 may prevent the smooth transition between tapered head 130 and sleeve 152, and allow for catching of skin and tissue 210 within gap 140, against distal end 153.

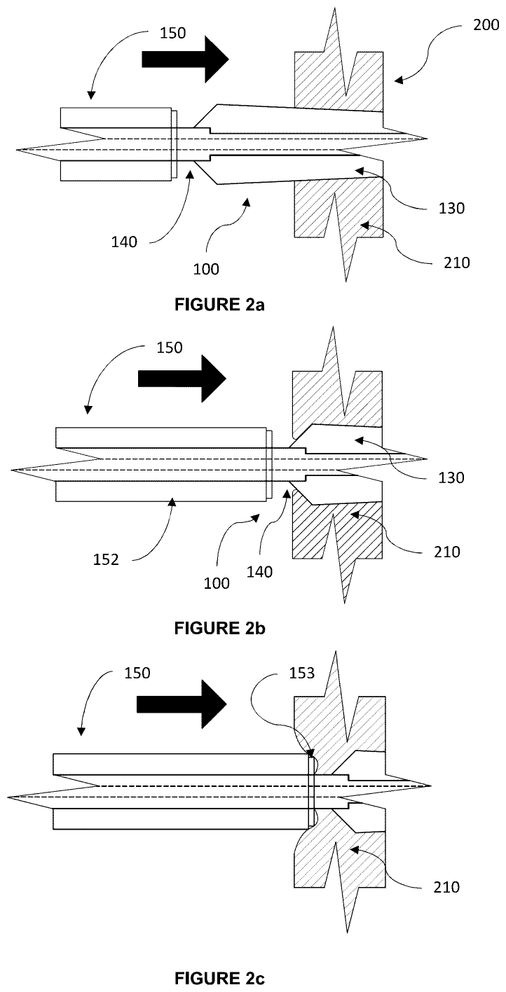

FIG. 2 provides front section views of the prior art dilator and sheath assembly shown in Detail A of FIG. 1 to provide a visual description of the prior art problem associated with the presence of gap 140. FIG. 2a shows the prior art dilator 100 and introducer sheath 150 assembly during initial insertion into an incision within the tissue 210 of patient 200. Tapered head 130 provides a smooth dilation of the incision within tissue 210, but manufacturing error produces gap 140 between tapered head 130 and distal end 153.

FIG. 2b shows a progressed insertion of the prior art dilator 100 and introducer sheath 150 assembly wherein the tapered head 130 has further inserted into tissue 210. Tissue 210 has a degree of elasticity which applies pressure on tapered head 130. At the proximal end of tapered head 130, when gap 140 is present, tissue 210 conforms to the shape of tapered head 130 and in the absence of a smooth transition between tapered head 130 and sleeve 152, enters gap 140.

FIG. 2c shows the resulting catching of skin and tissue 210 against distal end 153 of the prior art introducer sheath 150 at the incision point. The catching of skin and tissue 210 causes excess trauma or blocks further insertion of the prior art dilator 100 and introducer sheath 150 assembly. In some instances, either of these possibilities result in a failed insertion requiring removal of the dilator 100 and introducer sheath 150 assembly and a repeat attempt, which itself may increase trauma and contribute to the overall risk profile.

The invention described herein aims to ameliorate this problem, frequently associated with such prior art devices.

### An Embodiment According to The Invention

An embodiment is described in detail below with reference to the Figures. Exemplary embodiments are described to illustrate certain aspects and embodiments of the invention, not to limit their scope, which is defined by the claims. Those of ordinary skill in the art will recognize that a number of equivalent variations of the various features provided in the description that follows may be possible.

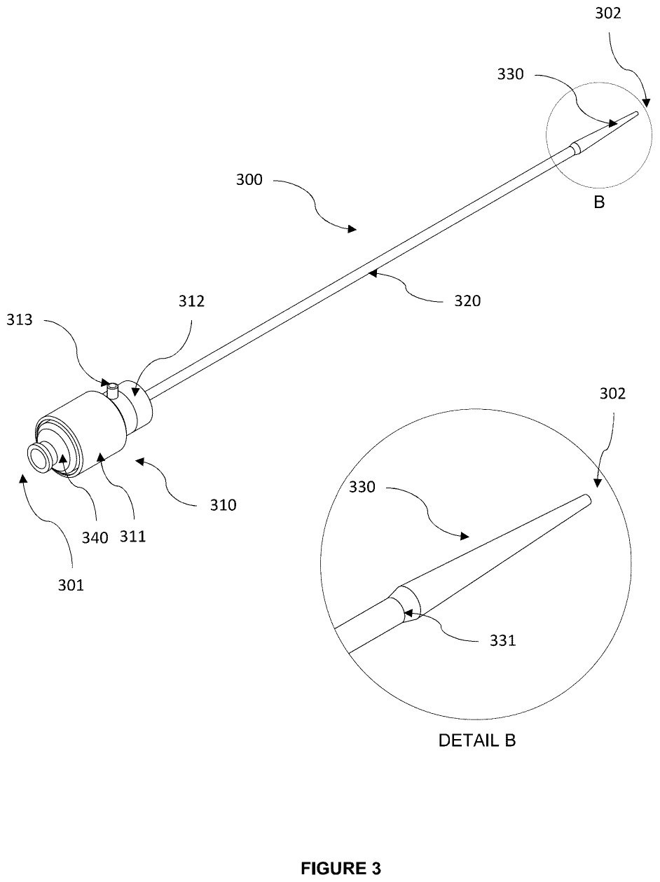

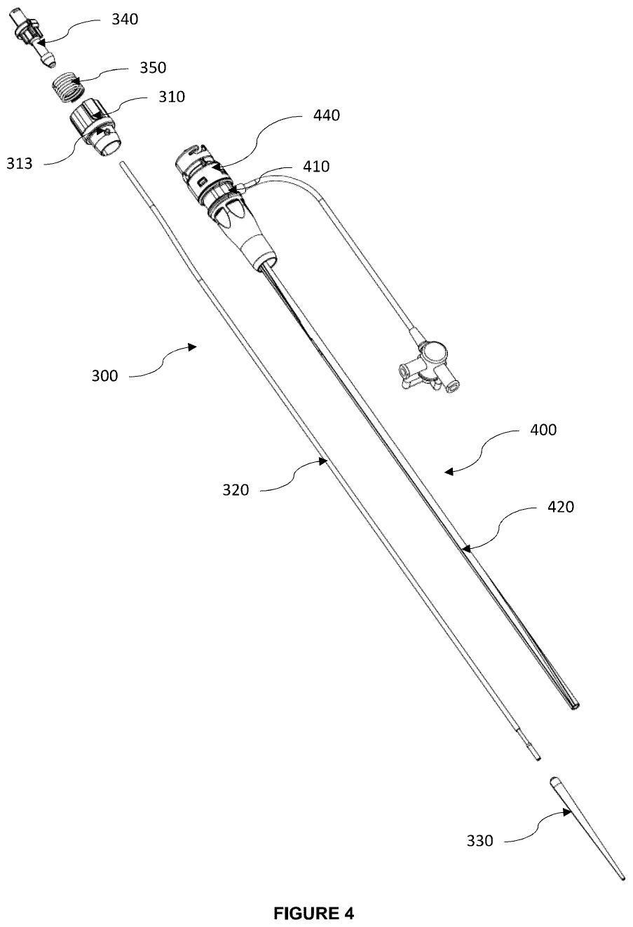

FIG. 3 provides a side perspective view of dilator 300. Dilator 300 is broadly constructed from a dilator hub 310 at its proximal end 301, a tapered head 330 at its distal end 302, and a dilator body 320 therebetween. A lumen from the proximal end 301 to the distal end 302 of dilator 300 allows wires and small instruments to be passed therethrough.

Dilator hub 310 provides an attachment point for an introducer sheath (not shown) and allows the operator to handle the dilator 300 during insertion of the dilator into the introducer sheath. Dilator hub 310 is a rigid annular structure having a larger diameter portion 311 and a smaller diameter portion 312. The smaller diameter portion 312 is shaped to fit within an introducer sheath collar. Dilator hub 310 further comprises a securing means embodied in a bayonet fitting 313 formed at the surface of the smaller diameter portion 313 of dilator hub 310 body, and a button 340 slidably inserted within the larger diameter portion 311 of dilator hub 310 and coupled thereto by a biasing element (not shown) embodied in a spring, which is attached at one end to the button 340 and the other end to the dilator hub 310.

Dilator hub 310, button 340, dilator body 320, and tapered head 330 contain a lumen therethrough such that wires and small instruments can be passed to the vessel. Detail B provides detail of tapered head 330, which is further used to ease insertion of dilator 300 into an introducer sheath. Tapered head 330 is substantially conical or arrowhead shaped and tapers outward from the distal end diameter of dilator body 320 at the interface 331 between tapered head 330 and dilator body 320, to the maximum diameter of tapered head 330, and tapers inward from the maximum diameter of tapered head 330 to a rounded apex at the distal end of tapered head 330.

The space between the interface 331 between tapered head 330 and the dilator body 320 and the maximum diameter of tapered head 330 provides a catch point for a patient's vessel when introduced with a sheath.

Dilator body 320 is substantially cylindrical and is attached at the distal end to tapered head 330 and at the proximal end enters dilator hub 310 where it is attached to button 340. The coupling between button 340 and the biasing element allows dilator body 320 to slidably move longitudinally relative to dilator hub 310.

The components of dilator 300 are constructed of a biocompatible polymer such as polycarbonate, polypropylene, Acrylonitrile butadiene styrene, polyethylene, polyamide or polyamide blend. Dilator body 320 may additionally incorporate metallic or synthetic braided reinforcement to provide increased rigidity. The components may incorporate additives or elements to improve opacity under x-ray imaging such as metallic components or fillers.

FIG. 4 provides a side perspective exploded view of the components of a dilator 300 and introducer sheath 400 assembly according to preferred embodiments of the invention. Dilator 300 broadly comprises the dilator hub 310, button 340, securing means 313, a biasing element embodied in a 0.1N/mm to 10N/mm conventional stainless steel coil spring 350, dilator body 320, and tapered head 330. Introducer sheath 400 broadly comprises a rigid annular collar 410, an elongated dual layer sleeve 420, and a securement fitting embodied in a bayonet receiver 440 suitable for receiving securing means 313 and securing dilator 300 thereto.

Introducer sheath 400 is broadly constructed of two main components; a rigid annular collar 410 and an elongated dual layer sleeve 420. Collar 410 is a hollow structure constructed to allow materials to be fed into the inner lumen of sleeve 420 through the wider opening of the sleeve 420. Collar 410 is formed of a rigid material to allow a user to handle the collar 410 and pass an object or material therethrough. Collar 410 comprises a securement fitting 440 and terminates in an opening shaped to enable sleeve 420 to be placed and secured therein, thereby connecting the two components.

Dilator hub 310 securably attaches to collar 410 by engaging securing means 313 to securement fitting 440 after insertion of dilator 300 into introducer sheath 400 to locate tapered head 330 relative to the distal end 430 of sleeve 420 and prevent further relative movement thereof.

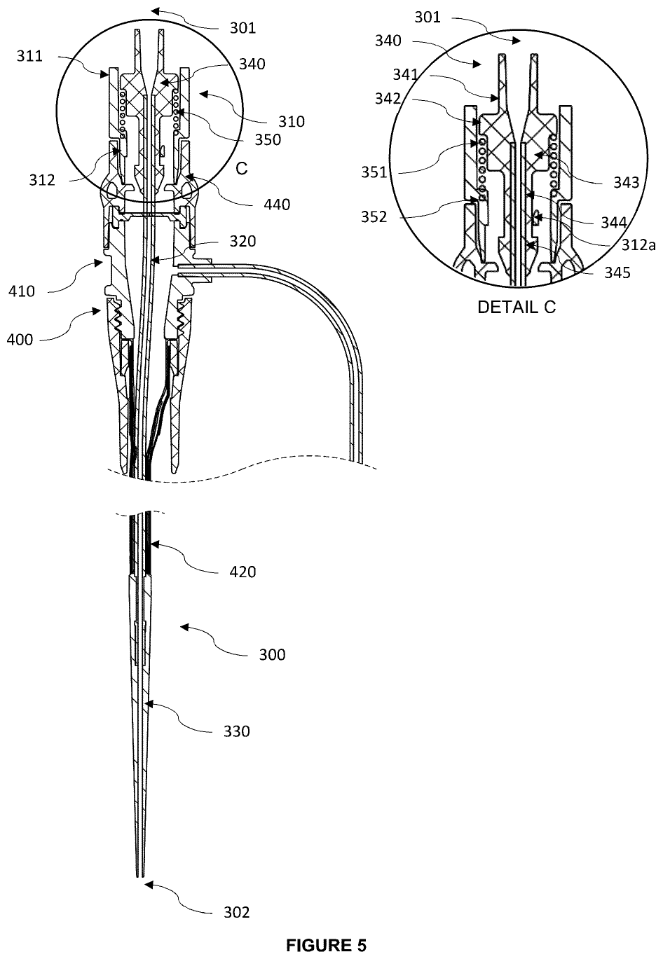

FIG. 5 provides a front section view of the dilator 300 and introducer sheath 400 in an assembled arrangement. The assembly of dilator 300 and introducer sheath 400 is such that dilator body 320 is enshrouded by the length of sleeve 420 which nominally abuts the proximal end of tapered head 330. Dilator hub 310 has a larger diameter portion 311 at its proximal end and a smaller diameter portion at its distal end 312 wherein the smaller diameter portion 312 is shaped to slidably insert into collar 410 and the larger diameter portion is shaped such that it then abuts the proximal end of collar 410.

Button 340 is formed as a unitary annular component shaped to broadly comprise a pusher 341 at its proximal end which is shaped to accept a syringe connection therein, an actuator core 343 connected thereto, a hook ridge 342 at the interface between the pusher 341 and the actuator core 343, a plunger body 344 at the distal end of actuator core 343, and a plunger stopper 345 at the distal end of button 340.

Pusher 341 is shaped for tactile engagement by a user to push button 340 slidably within larger diameter portion 311, with a central lumen therethrough to accept wires and instruments. Actuator core 343 is substantially cylindrical with a central lumen therethrough. The diameter of actuator core 343 provides a space between actuator core 343 and larger diameter portion 311 to fit spring 350. The hook ridge 342 is a portion of the actuator core 343 which is larger diameter and configured to slidably fit within larger diameter portion 311 and contain spring 350 within the space between actuator core 343 and larger diameter portion 311.

Plunger body 344 is substantially cylindrical with a central lumen therethrough. Its diameter is smaller than actuator core 343 and shaped to fit within smaller diameter portion 312 and extend therethrough. Plunger stopper 345 is shaped to strike a surface 312a inside smaller diameter portion 312 to maintain button 340 inside dilator hub 310.

Spring 350 is coiled spring steel having a coil proximal end 351 and a coil distal end 352. Spring 350 is sized to slidably fit within larger diameter portion 311 with a gauge sized to fit between actuator core 343 and larger diameter portion 311. Coil proximal end 351 abuts hook ridge 342 and imparts force thereon. Coil distal end 352 is attached at the interface between larger diameter portion 311 and smaller diameter portion 312 such that expansion is towards the coil proximal end 351.

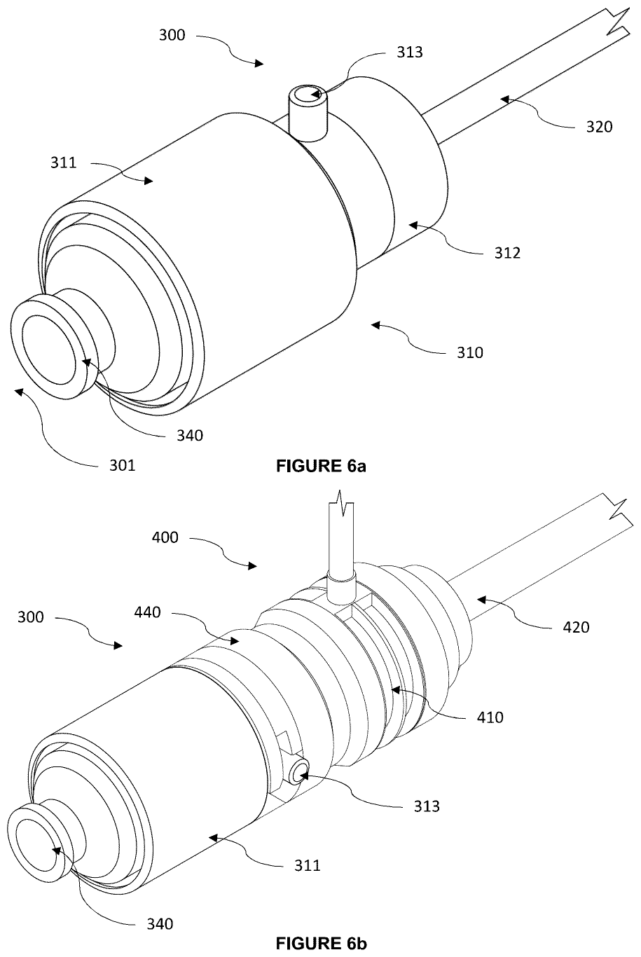

FIGS. 6a and 6b provide detailed side perspective views of the proximal components of dilator 300 and introducer sheath 400 according to the preferred embodiments of the invention illustrated in FIGS. 4 and 5.

As illustrated in FIG. 6a, dilator hub 310 is a rigid substantially tubular component having a larger diameter portion 311 at its proximal end and a smaller diameter portion 312 at its distal end, and containing a biasing element, embodied in a spring 350 (not shown), within and about the internal circumference of its larger diameter portion 311. Button 340 is slidably inserted within dilator hub 310 and abutting spring 350 such that further ingress into dilator hub 310 is resisted. Dilator body 320 is coupled to button 340 such that the actuation of button 340, as biased by spring 350, results in the longitudinal movement of dilator body 320, and tapered head 330 attached thereto towards distal end 302. Integrally formed with smaller diameter portion 312 of dilator hub 310 is a securing means, embodied as a bayonet type fitting 313, which allows dilator hub 310 to be secured to introducer sheath 400.

The securement of dilator 300 to introducer sheath 400 is detailed in FIG. 6b wherein a bayonet style fitting of securing means 313 is inserted into corresponding securement fitting 440 and secured thereto.

Sleeve 420 is dual layer, having an outer elastomeric layer applying outward resistance to a semi-rigid inner layer. Sleeve 420 is attached to rigid collar 410 at its proximal end. Securement fitting 440 is attached to the proximal end of collar 410 and provides a corresponding fitting for securing means 313. The secure engagement of securing means 313 to securement fitting 440 provides an anchor point allowing movement of dilator body 320 and tapered head 330 relative to sleeve 420 when button 340 is actuated and deactuated.

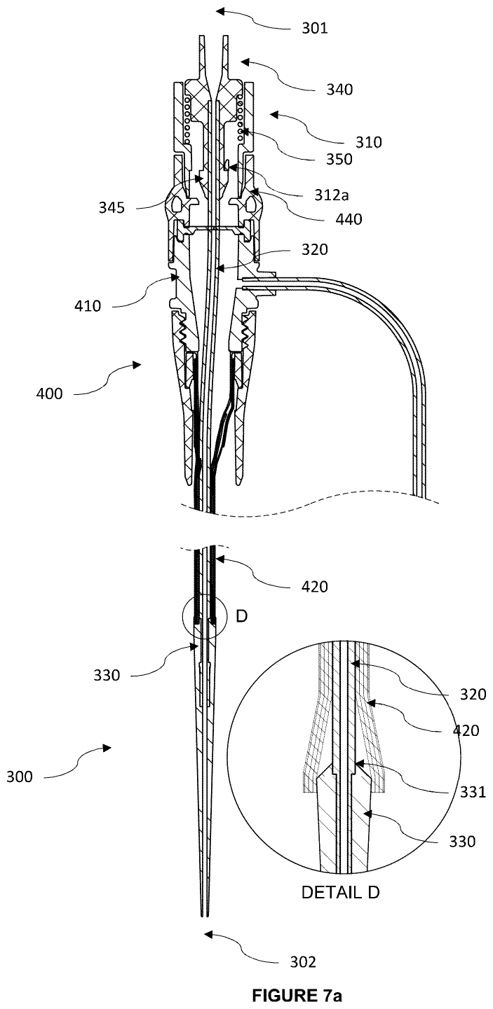

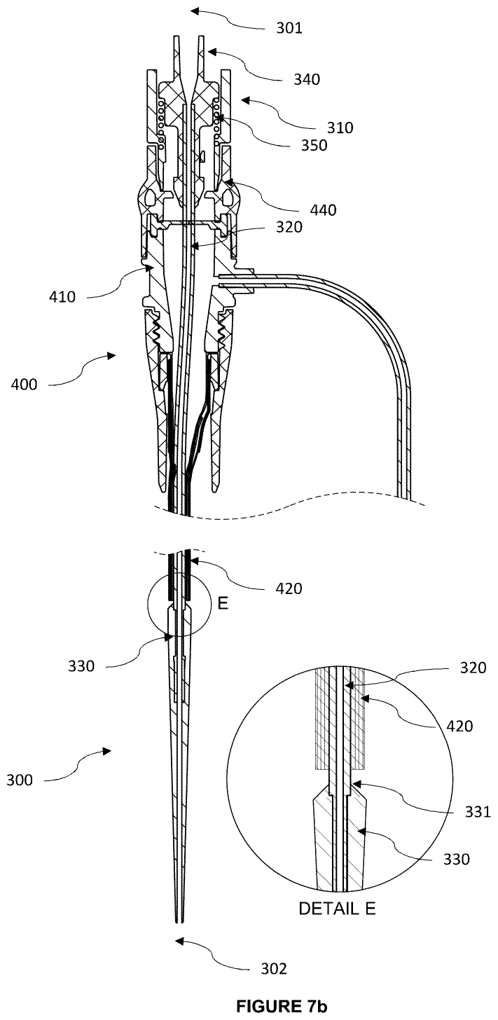

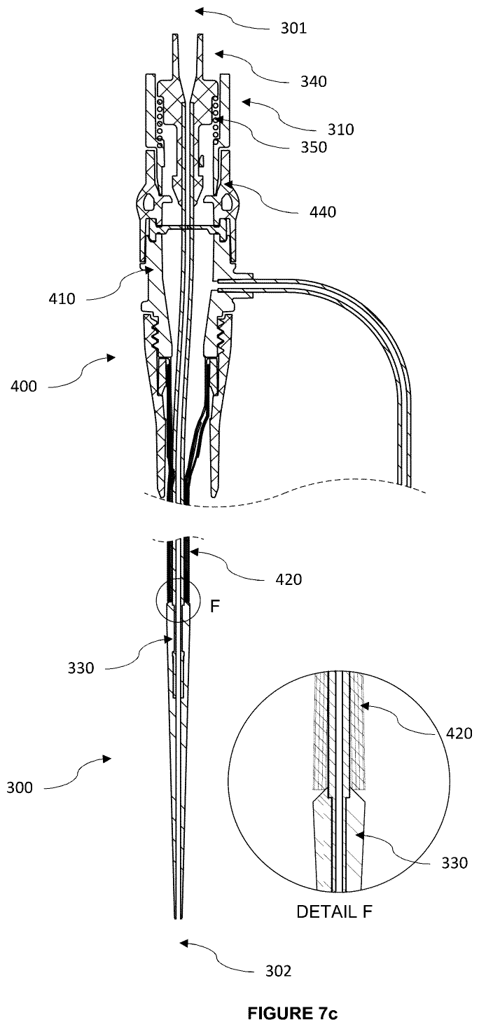

FIGS. 7a to 7c provide front section views of the dilator 300 and introducer sheath 400 wherein dilator hub 310 is attached to collar 410 via the engagement of securing means 313 to securement fitting 440 and details the tapered head 330 and sleeve 420 interaction at various points of actuation of button 340.

Once dilator 300 is inserted into introducer sheath 400, securement means 313 engages securement fitting 440 to secure thereto. Button 340 and dilator body 320 are coupled for combined longitudinal movement relative to introducer sheath 400. Button 340 is further coupled to spring 350 to allow combined longitudinal movement of button 340 and dilator body 320 towards the distal end 302 of introducer sheath 400 when the button 340 is actuated and provides a return force to the proximal end 301 when deactuated.

FIG. 7a provides a front section view of dilator 300 and introducer sheath 400 after the initial insertion of dilator 300 into introducer sheath 400 and the securement therebetween, but prior to actuation of button 340. Spring 350 is therefore in a substantially uncompressed state and plunger stopper 345 rests against surface 312a. The length of dilator body 320 is such that at this point tapered head 330 does not fully exit from the distal end of sleeve 420, as detailed in Detail D.

Pressing button 340 contracts spring 350 and pushes tapered head 330 beyond the distal end of sleeve 420. FIG. 7b provides a front section view of dilator 300 and introducer sheath 400 after the initial insertion of dilator 300 into introducer sheath 400 and the securement therebetween, and during the maximum actuation of button 340. Spring 350 is therefore in a state of maximum compression and dilator body 320 is at its maximum stroke. At this point, tapered head 330 has exited fully from the distal end of sleeve 420, and a gap is left therebetween, as detailed in Detail E.

Releasing button 340 allows spring 350 to expand and tapered head 330 to return and abut sleeve 420 at its distal end to produce a smooth transition therebetween. FIG. 7c provides a front section view of dilator 300 and introducer sheath 400 after the initial insertion of dilator 300 into introducer sheath 400 and the securement therebetween, and after release of button 340 from its maximum actuation to an intermediate point. Spring 350 is released from maximum compression to an intermediate position. At this point tapered head 330 has retracted back against sleeve 420 and is prevented from further retraction due to the rigidity of sleeve 420 and the low force exerted thereon by spring 350. The gap between tapered head 330 and sleeve 420 has been closed leaving a substantially smooth transition between tapered head 330 and sleeve 420, as detailed in Detail F.

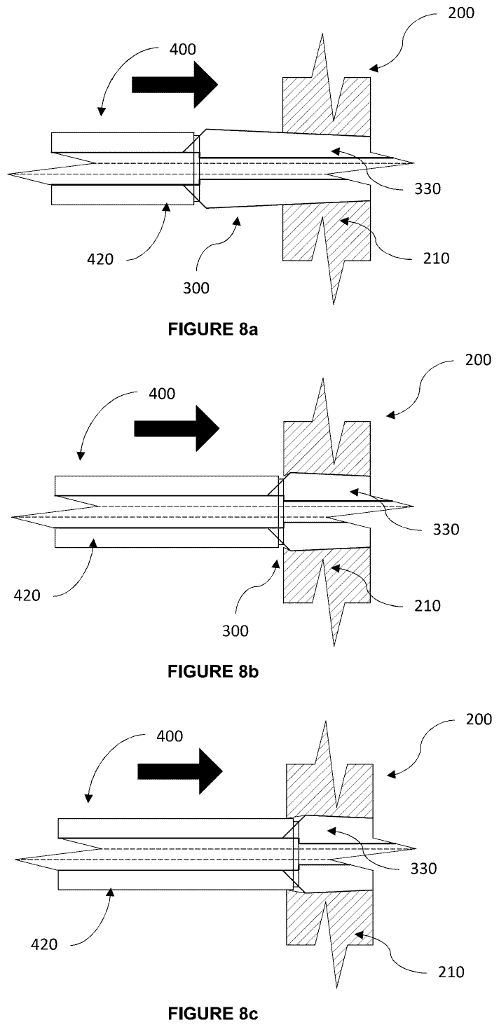

FIG. 8 provides front section views of the dilator 300 and sheath 400 assembly shown in Detail F of FIG. 7c to provide a visual description of the invention in use, wherein the dilator 300 and introducer sheath 400 assembly is inserted into an incision within the tissue 210 of a patient 200 after the button 340 has been actuated and deactuated to position tapered head 330 relative to sleeve 420. FIG. 8a shows the dilator 300 and introducer sheath 400, wherein tapered head 330 rests against the distal end of sleeve 420 due to the expansion force of spring 350, during initial insertion into an incision within the tissue 210 of patient 200. Tapered head 330 provides a smooth dilation of the incision within tissue 210 to reduce trauma.

FIG. 8b shows the initial interaction between the incision within tissue 210 and the tapered head 330 upon further insertion. Tissue 210 has a degree of elasticity which applies inward pressure on tapered head 330. At the proximal end of tapered head 330, when there is no gap present, tissue 210 conforms to the shape of tapered head 330 but is prevented from significantly entering the path of sleeve 420 by the substantially smooth transition therebetween.

FIG. 8c shows the resulting lack of catching of the patient's 200 skin and tissue 210 at the incision point against the distal end of sleeve 420. The substantially smooth transition from tapered head 330 to sleeve 420 allows tapered head 330 and sleeve 420 to enter the incision within tissue 210 without catching and causing excess trauma and subsequently reducing the risk of a failed insertion, thereby decreasing the patient's 200 overall risk profile.

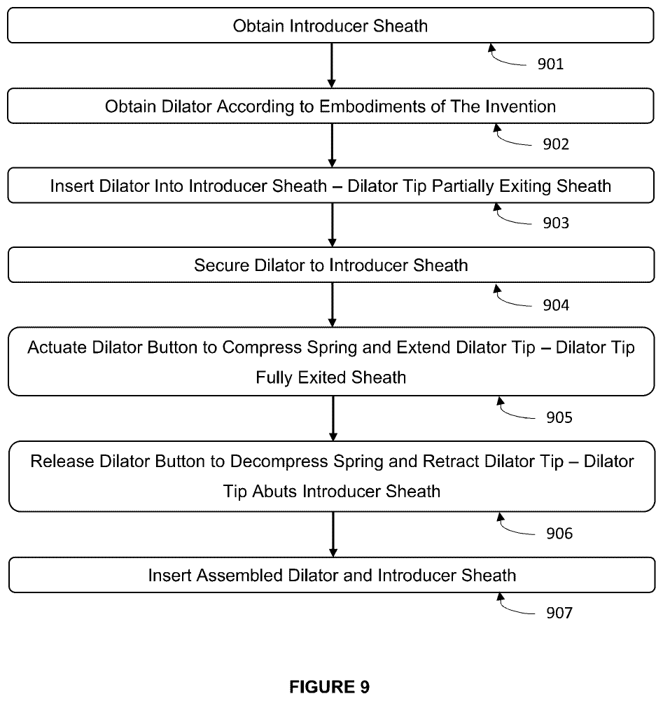

FIG. 9 provides a flowchart to demonstrate how the novel combination of integers illustrated in FIGS. 3 to 8c may be operated in regular usage scenarios. At Step 901, an introducer sheath suitable for assembly with a dilator according to embodiments of the invention is obtained, and at Step 902 the dilator itself is obtained.

Step 903 requires that the dilator tip is inserted into the proximal end of the introducer sheath such that when fully inserted, the dilator tip partially exits the distal end of the introducer sheath. Step 904 then requires the dilator to be secured to the introducer sheath in this position by engaging the securing means of the dilator with the securement fitting of the introducer sheath.

At Step 905 the user actuates or presses the button on the dilator to compress the spring coupled thereto, and fully extend the dilator tip relative to the introducer sheath. This has the effect of fully exposing the dilator tip and producing a gap between the dilator tip and the distal end of the introducer sheath.

At Step 906 the user deactuates or releases the button on the dilator allowing the spring to decompress and retract the dilator tip towards the proximal end of the introducer sheath. This has the effect of pulling the dilator tip against the distal end of the introducer sheath and closing the gap between the dilator tip and the introducer sheath to produce a substantially smooth transition therebetween.

At Step 907 the dilator and introducer sheath assembly according to embodiments of the invention is inserted into the patient as illustrated in FIGS. 8a to 8c.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

It will be understood that the terms ‘fastener’ or ‘fastening’, ‘coupling’ or ‘sealing’ when used alone or together with other terms such as ‘means’ or others, may be used interchangeably where interpretation of the term would be deemed by persons skilled in the art to be functionally interchangeable with another. Further, the use of one of the aforementioned terms does not preclude an interpretation when another term is included.

The various apparatuses and components of the apparatuses, as described herein, may be provided in various sizes and/or dimensions, as desired. Suitable sizes and/or dimensions will vary depending on the specifications of connecting components or the field of use, which may be selected by persons skilled in the art.

It will be appreciated that features, elements and/or characteristics described with respect to one embodiment of the disclosure may be used with other embodiments of the invention, as desired.

Although the preferred embodiments of the present disclosure have been disclosed for illustrative purposes, those skilled in the art will appreciate that various modifications, additions, and substitutions are possible, without departing from the scope and spirit of the disclosure and accompanying claims.

It will be understood that when an element or layer is referred to as being “on” or “within” another element or layer, the element or layer can be directly on or within another element or layer or intervening elements or layers. In contrast, when an element is referred to as being “directly on” or “directly within” another element or layer, there are no intervening elements or layers present.

As used herein, the term “and/or” includes any and all combinations of one or more of the associated listed items.

It will be understood that, although the terms first, second, third, etcetera, may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section could be termed a second element, component, region, layer or section without departing from the teachings of the present disclosure.

Spatially relative terms, such as “lower”, “upper”, “top”, “bottom”, “left”, “right” and the like, may be used herein for ease of description to describe the relationship of one element or feature to another element(s) or feature(s) as illustrated in the figures. It will be understood that spatially relative terms are intended to encompass different orientations of structures in use or operation, in addition to the orientation depicted in the drawing figures. For example, if a device in the drawing figures is turned over, elements described as “lower” relative to other elements or features would then be oriented “upper” relative the other elements or features. Thus, the exemplary term “lower” can encompass both an orientation of above and below. The device may be otherwise oriented (rotated 90 degrees or at other orientations) and the spatially relative descriptors used herein should be interpreted accordingly.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “including,” “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Embodiments of the description are described herein with reference to diagrams and/or cross-section illustrations, for example, that are schematic illustrations of preferred embodiments (and intermediate structures) of the description. As such, variations from the shapes of the illustrations as a result, for example, of manufacturing techniques and/or tolerances, are to be expected. Thus, embodiments of the description should not be construed as limited to the particular shapes of components illustrated herein but are to include deviations in shapes that result, for example, from manufacturing.

Unless otherwise defined, all terms (including technical and scientific terms) used herein have the same meaning as commonly understood by one of ordinary skill in the art to which this description belongs. It will be further understood that terms, such as those defined in commonly used dictionaries, should be interpreted as having a meaning that is consistent with their meaning in the context of the relevant art and will not be interpreted in an idealised or overly formal sense unless expressly so defined herein.

Any reference in this specification to “one embodiment,” “an embodiment,” “example embodiment,” etc., means that a particular feature, structure, or characteristic described in connection with the embodiment is included in at least one embodiment of the description. The appearances of such phrases in various places in the specification are not necessarily all referring to the same embodiment. Further, when a particular feature, structure, or characteristic is described in connection with any embodiment, it is within the purview of one skilled in the art to effect and/or use such feature, structure, or characteristic in connection with other ones of the embodiments.

Embodiments are also intended to include or otherwise cover methods of using and methods of manufacturing any or all of the elements disclosed above.

While the invention has been described above in terms of specific embodiments, it is to be understood that the invention is not limited to these disclosed embodiments. Upon reading the teachings of this disclosure many modifications and other embodiments of the invention will come to the mind of those skilled in the art to which this invention pertains, and which are intended to be and are covered by both this disclosure and the appended claims.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It is indeed intended that the scope of the invention should be determined by proper interpretation and construction of the appended claims and their legal equivalents, as understood by those skilled in the art relying upon the disclosure in this specification and the attached drawings.

1. A vascular dilator positioning assembly, comprising;
a grip portion comprising a wall having an outer wall surface and an inner wall surface, the wall having a proximal edge defining a proximal opening, a distal edge defining a distal opening and a lumen formed therethrough,
a user-operatable actuator located about the proximal edge, and
a vascular dilator comprising an elongated tube attached to the actuator at a proximal end and a dilator tip at a distal end,
wherein the actuator is configured to move the dilator tip distally from a retracted position to an extended position while the actuator is activated and is configured to return the dilator tip to the retracted position when the actuator stops being activated.

2. The vascular dilator positioning assembly according to claim 1, further comprising a resilient biasing element for biasing a dilator tip with respect to the wall.

3. The vascular dilator positioning assembly according to claim 2, wherein the resilient biasing element is a spring having a spring rate of between about 0.1 N/mm and 10 N/mm.

4. The vascular dilator positioning assembly according to claim 1, wherein the actuator has a button located about the proximal edge and an actuator core configured to engage with the wall, wherein the actuator is configured to move the dilator tip distally from the retracted position to the extended position upon the application of force to the button and is configured to return the dilator tip to the retracted position upon the release of force to the button.

5. The vascular dilator positioning assembly according to claim 4, wherein the actuator core is configured to occupy a space within the grip portion lumen.

6. The vascular dilator positioning assembly according to claim 4, further comprising a resilient biasing element for biasing a dilator tip with respect to the wall and wherein a portion of the actuator core is shaped to occupy a space across a width of the grip lumen thereby enabling the actuator to slide within the grip lumen, and
the resilient biasing element occupies a space substantially defined by an outer surface of the actuator core and the inner surface of the wall.

7. The vascular dilator positioning assembly according to claim 4, comprising;
a dilator sheath circumferentially surrounding the elongated tube having a proximal sheath end and a distal sheath end wherein the actuator is configured to move the dilator tip distally from a retracted position to an extended position upon the application of force to the button to widen a gap between the dilator sheath and the dilator tip and is configured to partially return the dilator tip to an intermediary resting position upon the release of force from the button thereby closing the gap between the dilator sheath and the dilator tip.

8. The vascular dilator positioning assembly according to claim 7, wherein the dilator sheath comprises a semi-rigid inner layer and an elastomeric outer layer and the dilator tip is configured to abut the semi-rigid inner layer at the intermediary position thereby providing a substantially smooth exterior surface across the elastomeric outer layer and the dilator tip. 9-13. (Cancelled)

14. A method of use of a vascular dilator positioning assembly, the method comprising the steps of:
obtaining a grip portion comprising a wall having an outer wall surface and an inner wall surface, the wall having a proximal edge defining a proximal opening, a distal edge defining a distal opening and a lumen formed therethrough and an user-operatable actuator located about the proximal edge,
attaching a vascular dilator comprising an elongated tube to the actuator at a proximal end and a dilator tip at a distal end,
placing a dilator sheath circumferentially around the elongated tube having a proximal sheath end and a distal sheath end, and
activating the actuator to move the dilator tip distally from a retracted position to an extended position to widen a gap between the dilator sheath and the dilator tip, and
stop activating the actuator to return the dilator tip to the retracted position to close the gap between the dilator sheath and the dilator tip.

15. The method of use of vascular dilator positioning assembly according to claim 14, further comprising the step of:
assembling the grip portion, the actuator and a resilient biasing element for biasing a dilator tip with respect to the wall.

16. The method of use of vascular dilator positioning assembly according to claim 14,
wherein the actuator has a button located about the proximal edge and an actuator core configured to engage with the wall,
wherein the step of activating the actuator comprises:
applying force to the button to move the dilator tip distally from a retracted position to an extended position to widen a gap between the dilator sheath and the dilator tip, and wherein the step of stop activating the actuator comprises:
releasing force to the button to partially return the dilator tip to an intermediary resting position thereby closing the gap between the dilator sheath and the dilator tip.

17. The method of use of vascular dilator positioning assembly according to claim 14, wherein the dilator sheath comprises a semi-rigid inner layer and an elastomeric outer layer and the dilator tip is configured to abut the semi-rigid inner layer at the intermediary resting position thereby providing a substantially smooth exterior surface across the elastomeric outer layer and the dilator tip.

18-20. (canceled)

21. The vascular dilator positioning assembly according to claim 1, wherein the dilator tip is conical with a taper towards a point at the distal end of the dilator tip.

22. The vascular dilator positioning assembly according to claim 7, wherein the distal sheath end abuts an interface between the elongated tube and the dilator tip.

23. The method of use of vascular dilator positioning assembly according to claim 15, wherein the resilient biasing element is a spring having a spring rate of between about 0.1 N/mm and 10 N/mm

24. A vascular dilator positioning assembly comprising:
a grip portion comprising a wall having an outer wall surface and an inner wall surface, the wall having a proximal edge defining a proximal opening, a distal edge defining a distal opening and a lumen formed therethrough;
a user-operatable actuator located about the proximal edge of the vascular dilator positioning assembly;
a vascular dilator comprising an elongated tube, and a dilator tip connected to the elongated tube at an interface, wherein a diameter of the dilator tip is greater than a diameter of the elongated tube;
a dilator sheath circumferentially surrounding the elongated tube, the dilator sheath having a proximal end and a distal end; and
wherein the actuator is configured to:
move the dilator tip distally from a retracted position to an extended position, with respect to the dilator sheath, upon activation of the actuator, widening a gap between the dilator tip and the distal edge of the dilator sheath, and
move the dilator tip proximally, upon deactivation of the actuator, such that the interface abuts the distal edge of the dilator sheath.

25. The vascular dilator positioning assembly according to claim 24, wherein the dilator tip is conical with a taper towards a point at the distal end of the dilator tip.
